(19) United States
(12) Patent Application Publication
(10) Pub. No.: US 2024/0057898 A1
(43) Pub. Date: Feb. 22, 2024

(54) INTRA-ORAL TEST DEVICE AND METHOD

(71) Applicant: Achaemenid, LLC, Stratford, CT (US)

(72) Inventor: Reza Radmand, Boston, MA (US)

(73) Assignee: Achaemenid, LLC, Stratford, CT (US)

(21) Appl. No.: 18/260,683

(22) PCT Filed: Jan. 7, 2022
(86) PCT No.: PCT/US2022/011536

§ 371 Date: Jul. 7, 2023

Related U.S. Application Data
(60) Provisional application No. 63/176,599, filed on Apr. 19, 2021.
(60) Provisional application No. 63/135,008, filed on Jan. 8, 2021.

(51) Int. Cl. A61B 5/145; A61B 10/00; A61B 5/00; A61B 5/1455

(52) U.S. Cl. CPC A61B 5/14507; A61B 10/0051; A61B 5/682; A61B 5/1455; A61B 5/14546

(57) ABSTRACT

A system for detecting target molecules in a patient's saliva includes an oral appliance and a cassette removably coupled to the oral appliance. According to an aspect, the cassette includes a microtubule including a collection portion. A detector is configured to transmit light through the removable cassette, measure the amount of light transmitted through the microtubule, determine an absorption or scattering amount of the light, and determine whether the target molecules are present in the patient's saliva based on the absorption or scattering amount of the light.

### CROSS-REFERENCE TO RELATED APPLICATIONS

This application is a 371 application of International Application PCT/US2022/011536 filed Jan. 7, 2022, which claims the benefit of and priority to U.S. Provisional Patent Application No. 63/135,008 filed Jan. 8, 2021, and U.S. Provisional Patent Application No. 63/176,599 filed Apr. 19, 2021, the entire contents of which are incorporated herein by reference.

### BACKGROUND

The hertz (symbol Hz) is a unit of frequency in the International System of Units (SI). It is defined as the number of cycles per second of a periodic phenomenon. The electromagnetic spectrum is the range of all possible frequencies of electromagnetic radiation. The infrared spectrum, (including near infrared, mid-infrared and far infrared), are segments of the electromagnetic spectrum with longer wavelengths than those of visible light, extending from a nominal red edge of the visible spectrum at 700 nanometers (nm) to 1 mm. This range of wavelengths corresponds to a frequency range of approximately 430 Terahertz (THz—1012 Hz) down to about 300 GHz (three hundred billion hertz). THz and sub-THz include segments of the electromagnetic spectrum between about far infrared and microwave, which have a wavelength of about 3 mm (three millimeters) to about 1 μm (one micrometer), corresponding to frequencies that range from about 100 GHz to 30 THz. These segments of the spectrum cannot be easily gauged with the optical and electronic measurement techniques normally associated with adjacent regions of the spectrum.

It is understood by those skilled in the art that it is possible to use the terahertz segment of the electromagnetic spectrum for diagnosis of dental disease or tissue changes in the hard and soft tissues of an intra and extra oral complexes. For example, use of the electromagnetic spectrum within the terahertz range is well established to diagnose dental decay more accurately and at an earlier stage than other ionizing x-ray techniques. This concept can also be used to illustrate multi-dimensional shapes of such anatomical structures using appropriate hardware and software to convert wave forms into identifiable anatomical images. Typically, the waves of this spectrum penetrate the tissue and reflect back to a detector, where they will be read and analyzed. This specific segment of the electromagnetic spectrum will only emit non-ionizing radiation, as compared to other conventional diagnostic tools such as the x-ray or gamma ray that can damage tissue cells.

Advances in technology have made possible the production and detection of infrared radiation with devices that are mobile and operate at room temperature. Perhaps the most commonly used generation method, in medical applications, employs optical rectification, whereby high frequency oscillations of a femtosecond laser pulse are rectified by an optical crystal, leaving only the envelope of the laser signal which is a THz pulse.

The air we breathe is typically polluted by various substances, such as dust, bacteria and viruses. For example, the virus that causes the novel coronavirus (COVID-19 or SARS Cov-2) may be transmitted through droplets generated when an infected person coughs, sneezes, or speaks. Current methods for testing whether a person is infected with the virus includes the insertion of a swab into the back of the nasal cavity through the nostrils or the back of the throat. The swab is rotated several times in order to obtain a sample of tissue and/or fluid from the nasal cavity and is then inserted into a container for sending to a laboratory for testing or by the use of rapid resting mechanism such as lateral flow testing method. Tests may be provided at hospitals or medical offices of healthcare providers; however, diagnostic tests are typically performed at testing sites where groups of people travel and wait in long lines to get tested or at home by the operator/patient. The results of the test are sent to a laboratory and can take from less than 24 hours to up to about 7 days. Rapid antigen test results currently available, can take approximately 15 minutes but are considerably less accurate (sensitive). The lateral flow rapid tests are typically not specific for SARS Cov-2 but only generic for SARS family of viruses.

Alternate SARS Cov-2 test methods, such as SARS Cov-2 rapid tests, require a person to expectorate into an external collection device, which may reduce the potential for capturing the proper quantity and/or quality of the microorganism, as this is considered merely a sample of the fluid collected at the particular time collection.

What is needed is a device and method for testing for the presence of molecular components of organisms such as viruses and bacteria in saliva. There is a further need for a diagnostic means that is mobile and suitable for personal use, encompasses use of a wide range of the electromagnetic spectrum, and can be used multiple times. There is a further need for a device that facilitates the capture of a clean and more abundant sample of oral fluids from a user without exposing the sample to the external environment.

### BRIEF SUMMARY

Embodiments of this disclosure are associated with a system for detecting target molecules in a patient's saliva. The system includes an oral appliance and a cassette removably coupled to the oral appliance. According to an aspect, the cassette includes a microtubule including a collection portion. A detector is coupled to the oral appliance, and is configured to transmit light through the cassette, measure the amount of light transmitted through the collection portion of the microtubule, determine an absorption or scattering amount of the light, and determine whether the target molecules are present in the patient's saliva based on the absorption or scattering amount of the light.

Further embodiments of the disclosure are associated with a system including an oral appliance, a cassette removably coupled to the oral appliance, and a detector in wireless communication with the cassette. It is contemplated that the system may be particularly suited for system for detecting target molecules in a patient's saliva. The cassette includes a microtubule having a collection portion. The cassette may be removed from the oral appliance, and thereafter positioned on a testing surface of the detector. According to an aspect, the detector is configured to transmit light through the collection portion, measure the amount of light transmitted through the collection portion, determine an absorption or scattering amount of the light, and determine whether target molecules are present in the patient's saliva based on the absorption or scattering amount of the light.

Additional embodiments of the disclosure are associated with a system for detecting target molecules in a patient's saliva. The system includes an oral appliance, and a cassette removably coupled to the oral appliance. According to an aspect, the cassette includes a microtubule including a collection portion. The cassette further includes a light transmitter configured to transmit light through the microtubule, and a detector configured to measure the amount of light transmitted through the collection portion. The detector may be further configured to determine an absorption or scattering amount of the light, and determine whether the target molecules are present in the patient's saliva based on the absorption or scattering amount of the light.

### BRIEF DESCRIPTION OF THE SEVERAL VIEWS OF THE DRAWINGS

A more particular description briefly described above will be rendered by reference to specific embodiments thereof that are illustrated in the appended drawings. Understanding that these drawings depict only typical embodiments and are not therefore to be considered to be limiting of its scope, exemplary embodiments will be described and explained with additional specificity and detail through the use of the accompanying drawings in which:

FIG. 1A is a perspective view of a typical mandible (lower jaw) of a patient for which the device and method are adapted, according to an embodiment;

FIG. 1B is a cross-sectional view of the mandible of FIG. 1A;

FIG. 2A is a perspective view of a stent, according to an embodiment;

FIG. 2B is a perspective view of a stent, according to an embodiment;

FIG. 3 is a perspective view of a bone density measuring device, according to an embodiment;

FIG. 4 is a perspective view of an alternative bone density measuring and diseased tissue detection device, according to an embodiment;

FIG. 5 is a plan view of a system for detecting target molecules in a patient's saliva, according to an embodiment;

FIG. 6 is a plan view of a system for detecting target molecules in a patient's saliva, according to an embodiment;

FIG. 7 is a side view of a cassette including a microtubule, according to an embodiment;

FIG. 8 is a side view of a cassette including a microtubule including nanoparticles, according to an embodiment;

FIG. 9 is a cross-sectional view of a microtubule including a collection portion and a treatment portion, according to an embodiment;

FIG. 10 is a cross-sectional view of the microtubule of FIG. 9, including a testing portion, according to an embodiment;

FIG. 11 is a side view of a microtubule including a covering member, according to an embodiment; and

FIG. 12 is a plan view of a system for detecting target molecules in a patient's saliva, according to an embodiment.

### DETAILED DESCRIPTION

Reference will now be made in detail to various embodiments. Each example is provided by way of explanation, and is not meant as a limitation and does not constitute a definition of all possible embodiments.

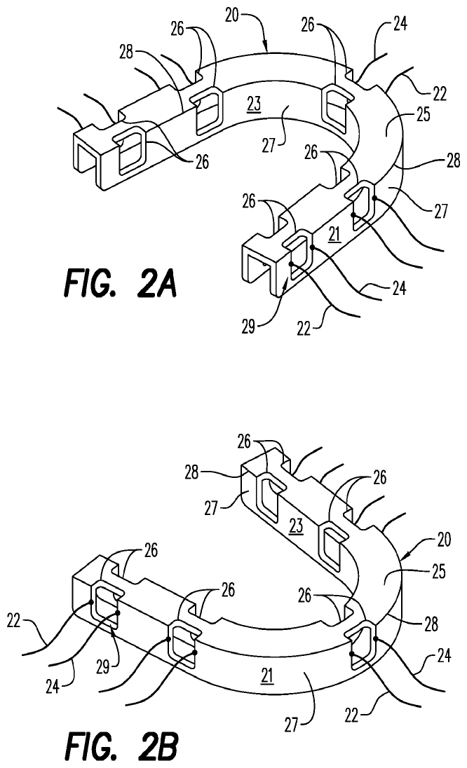

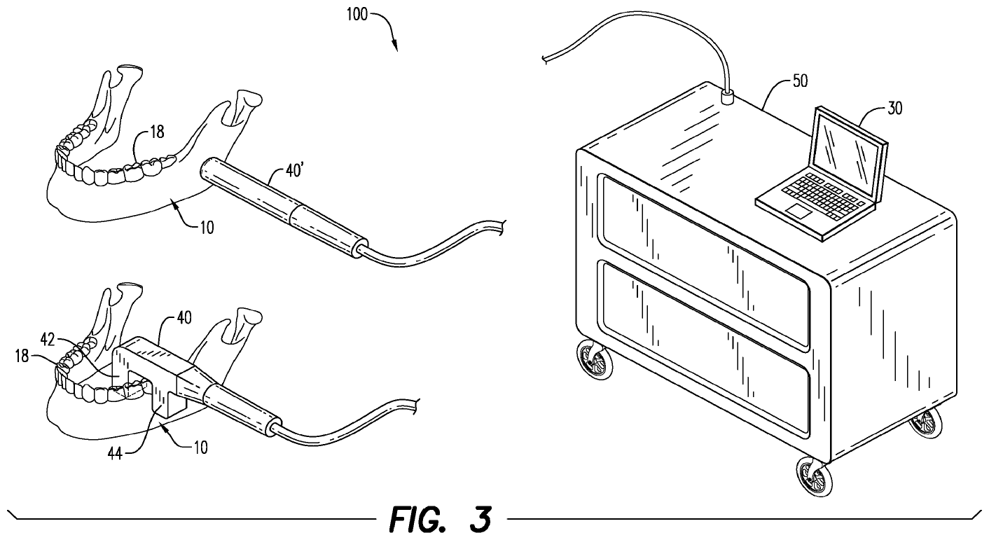

According to an aspect and with reference to FIG. 3, a diagnostic device 100 is provided that is configured for transmitting and receiving non-ionizing electromagnetic waves to measure the patient's hard and/or soft tissue abnormalities associated with the underlying hard and/or soft tissue. As described herein, components of one embodiment are intended for use in multiple embodiments, as would be understood by one of ordinary skill in the art. For instance, a computer 30 and/or spectrometer 50 depicted in FIG. 3, is capable of use with a stent 20 of FIGS. 2A and 2b, as will become more apparent with greater discussion hereinbelow.

In an embodiment the waves can be used to identify vascular changes occurring in a jawbone with satisfactory tissue differentiating abilities, as would be found, for instance in dental and/or systemic disease. According to a further aspect, infrared spectroscopy is used as a diagnostic tool for measuring blood perfusion within the jawbone (specifically Near Infrared Spectroscopy—NIRS). In yet another aspect, the terahertz wave is used to detect the soft and hard tissue changes. As used herein, the soft tissue is the tissue that connects, supports, or surrounds other structures and organs of the body, not being bone and includes but is not limited to gums, gingiva, intestinal tissue, vasculature, skin and the like, while the hard tissue includes but is not limited to bone, teeth, and the like.

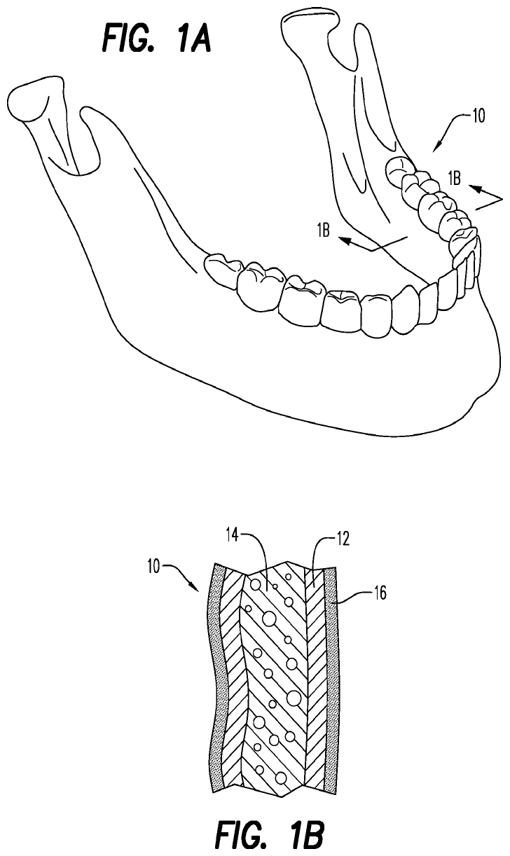

With reference to FIG. 1A, a mandible (a lower jaw or jawbone) 10 of a patient is shown. Although not specifically shown, embodiments described and shown herein that are operational with respect to the mandible are also applicable to the maxilla (upper jawbone).

With reference to FIG. 1B, a cross-sectional view of the mandible of FIG. 1A, the bone 10 includes a cortical bone 12 surrounding spongy bone 14, with the gums and soft tissue 16 covering the bone 10, which comprise hard and/or soft tissue as referenced herein.

Any comparative and relative hypo- or hyper-calcification within the medullary cavity associated with and/or incidental to dental and/or systemic disease, (which contains bone marrow (not shown)), for instance, shows itself as a decrease or increase in its density. Additionally, any reduction in bone perfusion within the medullary bone, may signify hyper calcification or increase in osteoblastic activity of the alveolar bone. A patient on a specific medication regimen that may change the density of the bone within short time intervals may need to be followed up closely as these changes occur. The use of electromagnetic radiation (i.e., THZ Time Domain, Infrared Fourier Transform), is particularly beneficial to patients who are receiving medications for osteoporosis or metastatic bone disease secondary to multiple myeloma, breast or prostate cancer, to name a few. The use of anti-resorptive medications is necessary to mitigate the loss of bone mass, leading to pathologic fracture of the bone.

In an aspect, it may be beneficial to repetitiously and accurately measure the changes occurring in the bone for at least the following reasons: 1) gauge the efficacy of these anti-resorptive drugs; 2) accurately follow and measure, in short time intervals, the incremental changes that occur in the jawbone during the administration of the anti-resorptive therapy, which according to current technology capabilities is not practical (the only current methods for bone density evaluation are ionizing radiation or MRI); 3) to develop an alternative protocol for drug administration, based on the changes that occur in the jawbone, hence reducing the incidence of osteonecrosis of the jaw (ONJ); 4) to detect the excessive and sometimes irreversible damage to the vital components of the bone complex (for example in the jawbone, this condition could lead to ONJ), with the aim to reduce or stop the use of these medications, in time, when such changes are detected; and 5) to evaluate the healing progression within the alveolar bone, post ONJ.

As discussed above, the full electromagnetic spectrum can be used to measure bone density/mass and bone vascular perfusion, as described herein. Since the infrared wave has a low tissue absorption rate, it is capable of penetrating through soft and hard tissue, up to several centimeters. The infrared wave is mainly absorbed by hemoglobin molecules, which aid in obtaining a value for the amount of vascular perfusion within the bone being measured. By measuring the vascular abundance in, for instance, the alveolar bone, a degree of osteoblastic activity, (bone deposition), during the administration of anti-resorptive therapy drugs or bisphosphonate medicines such as XGEVA® (denosumab) manufactured by Amgen Inc., to patients with metastatic bone disease (such as Giant Cell Tumor of Bone) and osteoporosis. Although these drugs are administered to counteract bone loss and generate tissue healing, in rare instances, death of bone cells and/or tissue result. Approximately 4-5% of patients taking such drugs experience a serious side effect of ONJ, also known as avascular necrosis of the jaw, resulting in drastic and unpleasant symptoms, including pain, inflammation of the surrounding soft tissue, secondary infection and/or drainage. The definitive sign of ONJ is the exposure of mandibular or maxillary bone through the gingiva over 8 weeks in duration. There may be no symptoms for weeks or months, until lesions with exposed bone appear. Use of the infrared wave and associated detection equipment as discussed in greater detail below for early detection and to measure the progressive diminishing of the alveolar vasculature can greatly benefit patients suffering from this debilitating disease.

Currently, as previously mentioned, the only way to closely monitor such changes is with the use of ionizing radiation and expensive MRI techniques.

According to an aspect, an apparatus and method for measuring hard and/or soft tissue abnormalities incidental to dental and/or systemic disease is provided.

In an embodiment, and with reference for instance to FIGS. 2a and 2b, a stent 20 is selected to overlay a patient's hard and/or soft tissue (see, for instance, FIG. 1A). According to an aspect, the stent 20 is selected based on the relative size of the mouth of the patient. According to an aspect, the stent 20 is customizable, that is, the stent 20 is created from impressions of each individual patient's mouth, and sized to overlay each patient's hard and/or soft tissue. As would be understood by one of ordinary skill in the art, depending on the particular circumstances of each individual patient, the lower and/or upper jaw may be limited in number of teeth 18 (including having no teeth), may have bone loss, and the like, when the stent 20 is customized.

With reference to FIGS. 2a and 2b, such an oral stent 20 is provided. In an embodiment, the overall dimensions and shape of the stent 20 may be formed through conventional dental molding techniques and are designed to fit over the teeth 18 (see FIG. 3) or edentulous jaw of the patient and lay in proximity to the gums 16 of the patient. In an embodiment, the stent 20 has a plurality of detection ports or openings 26, which are strategically positioned along the stent 20 and selected for positioning adjacent segments of the jawbone 10 having different bone density. As an example, an anterior portion of bone of the lower jaw 10 (FIG. 1A) may have a lower bone density than a posterior portion or segment of the bone, and is hence considered more vascular. The stent 20 typically includes an upper surface 25, flanked by sides 27 of the stent 20, along an edge 28.

In an embodiment, a pair of the plurality of detection ports 26 comprises a buccal-side 21 detection port 26 and a lingual-side 23 detection port 26. In an embodiment, the lingual-side detection port is positioned opposite the buccal-side detection port. In a further embodiment, the buccal-side 21 detection port is configured as a mirror image of the lingual-side 23 detection port. According to an aspect, the buccal-side 21 detection port is configured to transmit the electromagnetic wave, while the lingual-side 23 detection port is configured to receive data associated with the transmitted/received wave, and return data to an attached computer 30, which is configured for calculating, from the received data, the bone density and/or bone vascular perfusion adjacent and across each of the pairs of the plurality of ports 26. According to an aspect, the pairs of detection ports 26 are positioned along an edge 28 of the stent 20.

In an embodiment, the buccal side 21 of the stent 20 includes wires 22/24 connected to outer edges of each buccal-side port 26 as shown in FIGS. 2a and 2b for initiation of transmission of the electromagnetic wave and for receiving data related to measured bone density. In an embodiment, a wire in 22 is connected to a posterior edge of port 26, which is positioned on the buccal side 21 of the stent 20, while a wire out 24 is connected to an anterior edge of the same port 26. Positioned on the stent 20 opposite to the wires 22, 24, the port(s) 26 positioned on the lingual side 23 may include, for instance, a detector or sensor for receiving the transmitted wave (not shown). The plurality of ports 26 are capable of transmitting the sensed or measured IR wave related to the bone density and feeding the data back to a computer for analysis (as discussed with greater detail with reference to FIG. 3 hereinbelow).

In an embodiment, the wires 22, 24 are connected to a computer 30 and/or a spectrometer (see FIG. 3) or alternatively, a wireless connection may be made, which initiates a wave having a length and/or frequency in the electromagnetic spectrum described herein that ranges from the infrared range to the THZ range (approximate frequency range of 1011-1014 Hz) and transmits the wave from, for instance, the buccal side of the stent 20 through the bone across the port 26, and detects the wave to create an image of the bone density. The wires 22/24 are thus configured for transmitting the electromagnetic waves and receiving data associated therewith. According to an aspect, the wires 22/24 are attached at a first end 29 of the wires 22/24 to the buccal side ports 26, and attached at a second end to the computer 30 (not shown). In an embodiment, the diagnostic device is configured to transmit the electromagnetic waves from the buccal-side detection ports 26, through the patient's bone, and the lingual-side detection ports 26 being configured to receive the electromagnetic waves, such that changes occurring in the bone density and/or bone vascular perfusion are repetitiously and accurately measured, wherein the electromagnetic waves being infrared or terahertz electromagnetic waves.

In an embodiment, use of a portable THZ or IR (Infrared) device will allow a flat surface or a double surface probe 40 to be placed on the skin or gum tissue, over the lower or upper jawbone, through a stent 20 that fits securely, repetitiously, and consistently over the upper or lower jaw.

In an alternative embodiment and as seen in FIG. 3, the electromagnetic waves are transmitted/detected to/from a diagnostic probe 40, 40′ from a spectrometer 50, configured to measure electromagnetic waves having a frequency of between about 100 GHz to about 430 THz, or any portions or segments thereof. As shown herein, a flat surface diagnostic probe 40′ can be placed in proximity to the jawbone 10, and/or a double or multiple surface diagnostic probe 40 having a plurality or arms. As shown in FIG. 3, the probe 40 has a first arm 42 and a second arm 44, which can be placed in proximity to the teeth 18 or bone 10, such that the first arm 42 of the probe 40 extends along a lingual surface of the teeth 18 or bone 10, while an opposite arm or second arm 44 extends along a buccal surface of the teeth 18 or bone 10. In other words, the plurality of arms 42, 44 of the probe 40 straddle opposing sides of the teeth 18 of bone 10. The computer 30 is configured to receive the electromagnetic “fingerprint” spectra waves transmitted through the bone and to detect minute changes in the density of the bone, over the course of treatment using the anti-resorptive therapeutics.

Although not shown, it is further contemplated herein that the probe 40, 40′ could be used in conjunction with a stent 20. In such an embodiment, and as would be understood by one of ordinary skill in the art, the stent 20, which in an embodiment, has been customized for the particular, individual patient, would be used by the practitioner for locating the individual areas of the jaw to be measured.

It is well understood by those of ordinary skill in the art that the use of ionizing radiation is cumulative and harmful when used numerous times over a long period of time. Hence, according to an aspect, the patient may benefit from an alternate technique, which uses non-ionizing electromagnetic waves that can be used multiple times without the harmful side effects of the ionizing radiation such as X-rays and gamma rays. The shorter wavelengths of the infrared band will result in more clear spatial resolution and are therefore more accurate for diagnosis and identification of these subtle tissue changes. There have been many in vitro experiments in dentistry to determine the characteristics of enamel and dentin in detecting changes that can occur in these tissues in early dental caries.

In soft tissue studies, THz radiation was able to differentiate between the normal versus the diseased tissue even when compared with a conventional in vitro histological test. The objective according to an embodiment is to diagnose the extent of the diseased tissue (cancer of the oral soft tissue or skin) and in the process, conservatively remove as little of the surrounding healthy tissue as possible. An abnormal oral tissue (tongue, gum, inside of the cheek) to the naked eye or touch, can be evaluated first with the use of infrared radiation, which can distinguish quite accurately between normal tissue and abnormal or cancerous tissue. In an embodiment, the invasive nature of diseased tissue can also be detected, in an effort to establish a more conservative yet accurate approach to its treatment.

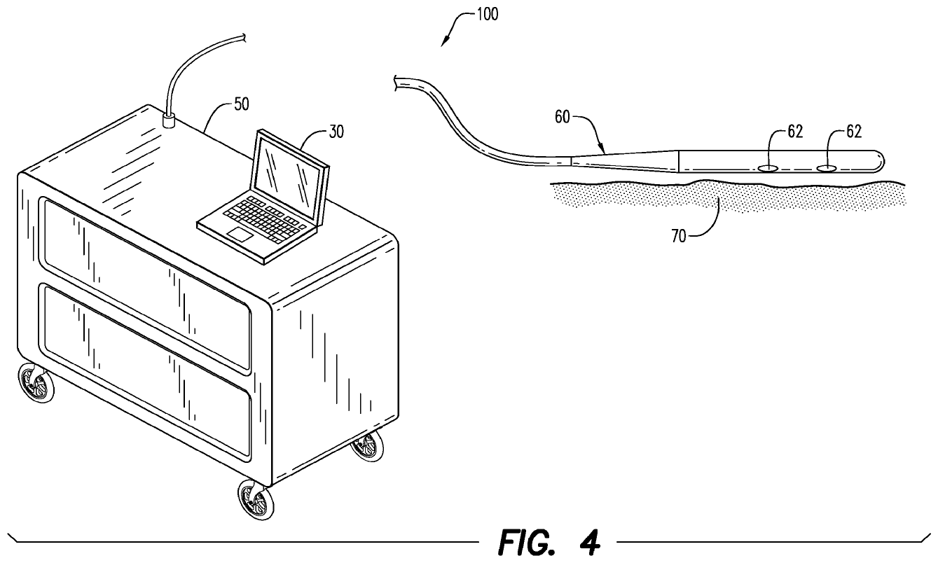

Similarly, according to an aspect, this technology can be used to view the internal tissue integrity such as tonsillar, genitourinary and the upper or lower gastrointestinal passages. In this alternative embodiment as shown in FIG. 4, the THz spectrometer 50 transmits/receives waves through an endoscopic probe 60 to detect unhealthy tissue 70 endoscopically. In this embodiment, the flexible endoscopic device with THz probe arrangement 60 is used, and the internal tissue 70 surfaces are scanned with the use of the THz radiation and fed back to the spectrometer machine 50 for analysis and diagnosis in a 3D format. In an embodiment, the endoscopic probe 60 has a plurality of transducers 62 arranged along the surface of the probe 60 for transmitting and detecting the infrared waves to detect tissue abnormalities.

According to an aspect, computerized diagnostic three-dimensional illustrative models can be generated, which can be used to plan for surgical procedures. Examples whereby such models would be beneficial include those in the field of craniofacial surgery in congenitally deformed patients, orthognathic surgery (surgery to correct conditions of the jaw and face related to structure, growth, sleep apnea, TMJ disorders, malocclusion problems owing to skeletal disharmonies, or other orthodontic problems that cannot be easily treated with braces) or dental implant placement in the jawbone. Additionally, in head and neck cancer therapy, for example, prior to elimination of the diseased tissues, it is essential to delineate the extent of pathology in the affected area. Subsequent surgery and/or ionizing radiation therapy to the region is then necessary to treat the disease. THz radiation can be instrumental in planning a conservative mapping of the diseased area in an attempt to identify the extent of pathology and minimize damage to the healthy surrounding tissue. In a further embodiment, the computer is configured to evaluate progression of tissue healing, secondary to ONJ.

Further embodiments of the disclosure may be associated with a system 100 configured to test a user's saliva for the presence of molecular components. The system 100 may be configured to test for the presence of proteins, enzymes, drug molecules and byproducts and various biomarkers. According to an aspect, the system 100 is configured to test for the presence molecular components of organisms such as viruses, bacteria, and the like. The system 100 may detect the presence of the genetic material of viruses and bacteria. This eliminates the need for a user to spit in a cup when at, for example, a healthcare provider's office or at home.

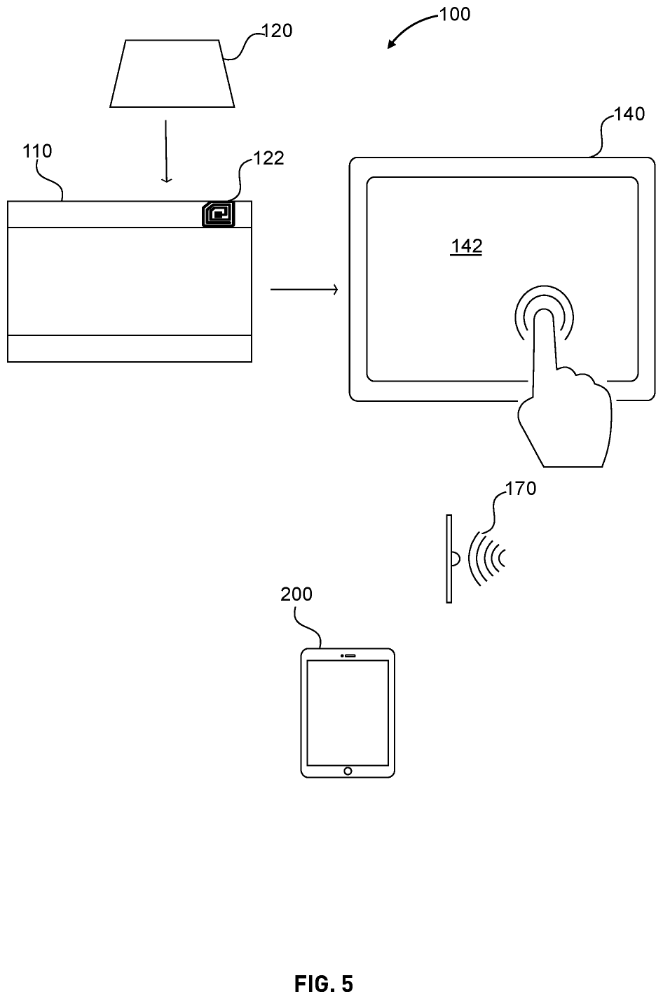

FIG. 5 is a plan view of the system 100 for detecting a target molecule 101 in a patient's saliva, according to an embodiment. According to an aspect, the target molecule 101 is an amino acid. As would be understood by one of ordinary skill in the art, various amino acids absorb light at different wavelengths. It is contemplated that the use of the system 100 defined herein to test for the presence of certain amino acids, for example, would be particularly suited for personal use and would eliminate the requirement for a user to expectorate into a cup or use nasal swabs to test for the presence of the genetic material of viruses and bacteria, or their amino acids, which would be indicative of the user being infected with such viruses, bacteria or other such organisms.

The system 100 includes an oral appliance 110 and a cassette 120 removably coupled to the oral appliance 110. According to an aspect, the cassette 120 is positioned into a housing (not shown) of the oral appliance 110 by the use of a fastening mechanism. The fastening mechanism may include one-way locking clips. The one-way locking clips may help to secure the cassette 120 in the oral appliance 110. According to an alternative aspect, the cassette 120 is secured to the oral appliance 110 by an adhesive or any other locking mechanism.

FIG. 5 illustrates an embodiment in which the cassette 120 is removably coupled to the oral appliance 110. Once coupled to the oral appliance 110, the cassette 120 communicates with a sensor 122 that is secured to the oral appliance 110. The sensor 122 may sense the presence of collected saliva in the cassette 120. Once the sensor 122 detects the presence of saliva, an alert may be sent to the user so that the saliva can be tested for the presence of the target molecule 101.

The oral appliance 110, with the cassette 120 secured thereto, may be positioned on a detector 140 or a light reader. According to an aspect, the oral appliance 110, with the cassette 120 secured thereto, is positioned on a testing surface 142 of the detector 140. The detector 140 transmits light through the cassette 120, measures the amount of light transmitted through the cassette 120, determines an absorption or scattering amount of the light, and determines whether the target molecule 101 is present in the patient's saliva based on the absorption or scattering amount of the light. For example, when a target molecule 101 includes a ribonucleic acid (RNA) of a virus, and the RNA is present in the user's saliva and collected in the cassette 120, the sample in the cassette 120 is tested using the detector 140.

According to an aspect, the detector 140 includes a photo-spectrometer/spectrophotometer. According to an aspect, the spectrophotometer may be utilized to obtain an image of the target molecule 101. The spectrophotometer includes a light transmitter 135 or light source. According to an aspect, the spectrophotometer may include a plurality of individual light transmitters 135. The spectrophotometer may further include an optical means that directs the light originating from the light transmitters 135 onto the sample of saliva to be tested.

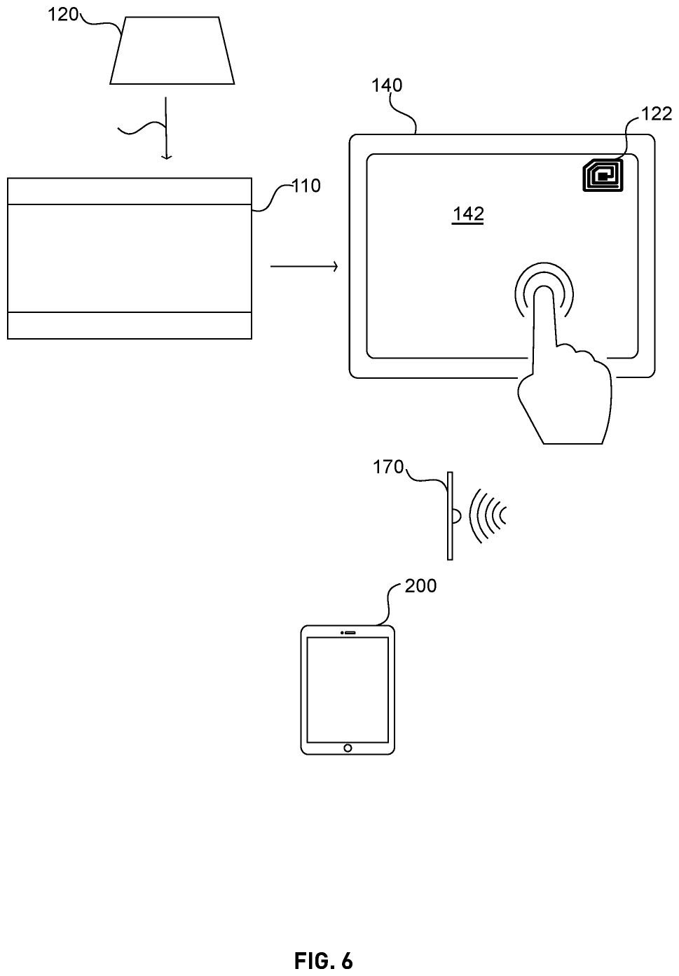

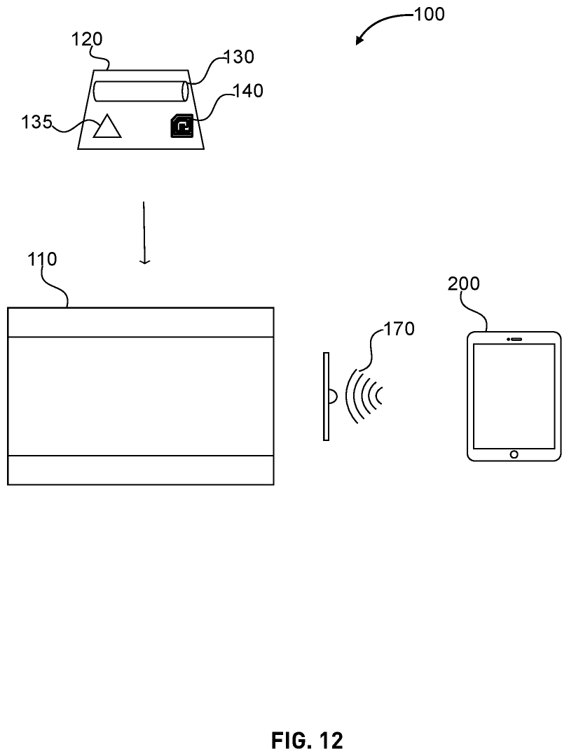

While only one cassette 120 is illustrated in FIG. 5, FIG. 6 and FIG. 12, it is contemplated that the system 100 may include more than one cassette 120. Each cassette 120 may be particularly suited for being arranged in or on the oral appliance 110, which includes a mouthpiece that is suitable for being positioned in the user's oral cavity and removably secured to the user's dentition. Each cassette 120 may be configured to test for the presence for a different target molecule 101.

According to an aspect, and as illustrated in FIG. 6, the sensor 122 may be included in the detector 140. In this configuration, once the oral appliance 110, with the cassette 120 secured thereto, or just the cassette 120, is positioned on the testing surface 142 of the detector 140, the sensor 122 can detect whether the cassette 120 includes an adequate quantity of the patient's saliva. If an adequate amount of saliva is present, then the detector 140 will be activated to transmit light through the cassette 120 to test the user's saliva. Based on the light absorption data that is obtained, the presence or absence of the target molecule 101 can be determined.

According to an aspect, the oral appliance 110 includes a customizable/custom mouthpiece. According to an aspect, the cassette 120 may be configured for being housed on or in a portion of the mouthpiece. The oral appliance 110 may include a pocket within which the cassette 120 may be positioned. The pocket may be made from the same material as the mouthpiece. It is contemplated that the cassette 120 may be arranged at any location on the mouthpiece. For example, the cassette 120 may be arranged at the lingual (tongue) side or the buccal (cheek) side of the mouthpiece.

The mouthpiece may be fabricated by either digitally scanning the user's jaw/dentition or taking conventional impression molds. A clear retainer type aligner may initially be fabricated on the dentition to help maintain the position and alignment of the mature dentition during oral appliance sleep therapy. It is contemplated that if the user is interested in performing smile line orthodontic treatment, orthodontic clear aligners can be fabricated and changed at timed intervals, such as every two weeks, while continuing with any prescribed therapy. The mouthpiece may first be molded and fitted on to the lower jaw using a thermoplastic “boil and bite” material or molded dental silicone liner material.

After scanning or using impression material to capture the dentition, the laboratory may fabricate custom oral appliance trays (e.g., top and bottom trays) that may include a cured and/or adhered lining that can be inserted over the already fabricated clear orthodontic aligners. It is contemplated that the oral appliance 110 may include only a bottom tray.

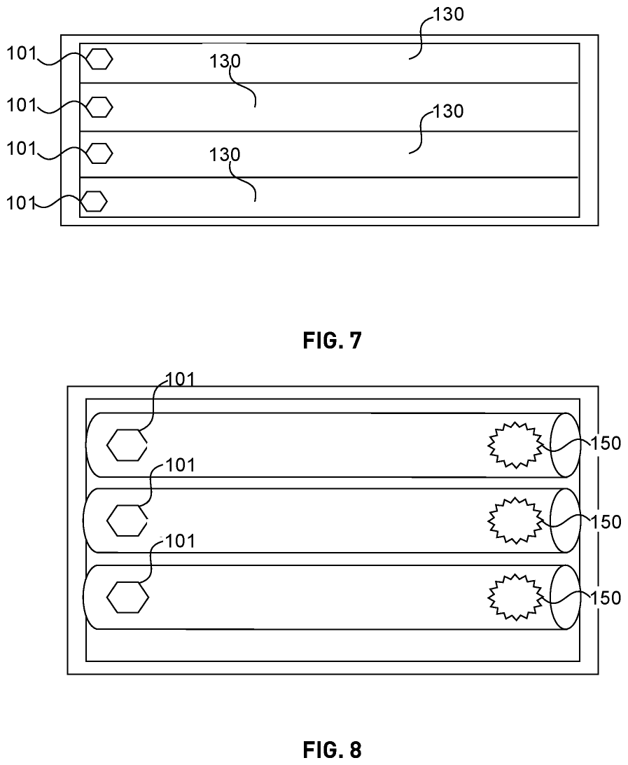

According to an aspect and as illustrated in FIG. 7 and FIG. 8, the cassette 120 includes a microtubule 130. The microtubule 130 is configured to receive the sample of the user's saliva, and will include the target molecule 101 if the user's saliva includes the target molecule 101. As illustrated in FIG. 7, the cassette 120 may include a plurality of microtubules 130. Each microtubule 130 may be configured to receive a sample of the user's saliva. According to an aspect, even if only one microtubule 130 of the plurality of microtubules 130 captures a sample of the user's saliva, that one microtubule 130 will include a sufficient quantity of the user's saliva for testing purposes.

According to an aspect, a plurality of microtubules 130 is embedded within the cassette 120. The plurality of microtubules 130 may be connected in parallel. In an embodiment (not shown), the microtubules 130 are arranged so that they are connected with each other, in series so that the saliva sample may travel from one microtubule to another microtubule. An array of the microtubules may be arranged in the cassette 120 so that the microtubules 130 are arranged at a clear or see-through portion of the cassette 120 to facilitate the transmission of light through the microtubules 130.

The microtubule 130 of FIG. 7 may be configured to only capture the user's saliva to test for the presence of the target molecule 101. In an alternate configuration, the microtubule 130 arrangement illustrated in FIG. 8 may be configured to combine the target molecule 101, if present, with a nanoparticle (NP) 150 or a plurality of nanoparticles (NPs) 150 (NP bio-clusters). When the target molecule 101 is combined with or adhered to the nanoparticles 150, via an ionic bond or a hydrogen bond, the detector 140 will detect whether the combination is present in the cassette 120. For example, if the target molecule 101 is an RNA molecule, the negative charge on the RNA molecule may be attracted to positive ions of the nanoparticles (such as positively charged gold nanoparticles). For example, the surfaces of the nanoparticles 150 can also be modified to target or determine the presence of the specific target molecule 101.

It is contemplated that a terminal end of the microtubule 130 may include biocompatible nanoparticles (NP bio clusters). Such biocompatible NP bio clusters may include silica, gold or silver clusters coated with a molecule that is specific for the particular molecule or organism that is to be detected, that is the target molecule 101. The use of gold, for example, may be advantageous because gold is inert and will not alter the composition of the user's saliva (for example, organic material in the user's saliva). Similarly, silica has positive compatibility with organic material. Silver has also been found to be bio compatible and will not negatively reaction with the target molecule 101. These NP bio clusters may be efficient at absorbing or scattering light. By changing their size, shape and composition, the optical response can be tuned from visible light to near-infrared.

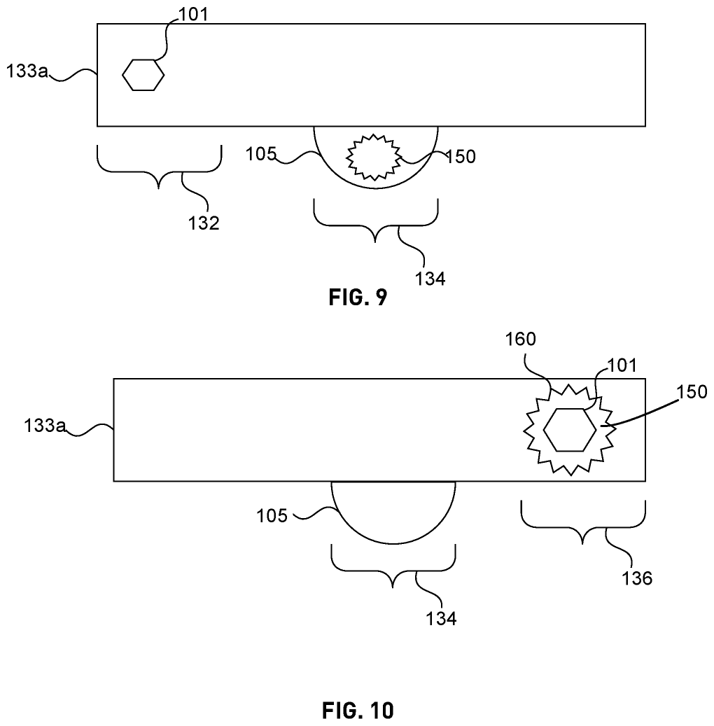

FIG. 9 and FIG. 10 illustrate the microtubule 130 in more detail. As illustrated in FIG. 9, the microtubule 130 may include a collection portion 132. The collection portion 132 may be any designated portion of area of the microtubule 130 that facilitates the entry of saliva within the microtubule 130. For example, the collection portion 132 may include an opening 133a that facilitates entry of the sample of saliva into the microtubule 130.

The microtubule 130 may further include a treatment portion 134 that is spaced apart from the collection portion 132. According to an aspect, the treatment portion 134 includes a pocket 105 within which a plurality of nanoparticles 150 are housed. The pocket 105 may be configured to house or at least temporarily retain the nanoparticles 150.

The nanoparticles 150 housed within the pocket 105 may include at least one of gold, silver and silica. According to an aspect, the nanoparticles 150 includes a compound including at least one of gold, silver and silica. The target molecule 101 may bind with at least one of the nanoparticles 150 to form a NP-bonded particle or target nanoparticles (target-NPs) 160. The target molecule 101 may move along a length of the microtubule 130 via capillary movement or hydrostatic pressure to bind with the nanoparticles 150 to form the Target-NP 160.

According to an aspect and as illustrated in FIG. 10, the microtubule 130 further includes a testing portion 136. The testing portion 136 may be spaced apart from the collection portion 132 and the treatment portion 134. In an embodiment (not shown), the testing portion 136 is integrated with the treatment portion 134. The Target-NP 160 may be housed in the testing portion. When the cassette 120 is positioned on the testing surface 142 of the detector 140, the light is transmitted through the Target-NP 160 or the saliva sample of the user to test for the presence of the target molecule 101.

According to an aspect, the target molecule 101 in the user's saliva may be biochemically primed to allow for its exposure to the NP bio clusters, specific for that target molecule 101. The biochemical primer may be included in the cassette 120, or specifically in the microtubule 130, in advance of the target molecule 101 reaching the NP bio clusters, used to identify the target molecule 101.

For example, the target molecule 101 may be the SARS-Cov 2 antigen. For the SARS-Cov 2 antigen to be detected in the saliva, the NPs are coated with the specific molecule for the SARS Cov2 virus. The system 100 may use a specific molecule attached to the NPs to detect the exact genetic material or protein that is part of the genetic sequence of SARS-Cov 2. When the biomarker binds to the virus's gene sequence, the particular SARS Cov-2 virus is identified. In the case of any genetic mutations of the organism, such as the SARS Cov-2 virus, the system 100 can specifically identify each mutation, based on the light absorption and/or light scattering fingerprint of the biomarker attached to the NP bio cluster.

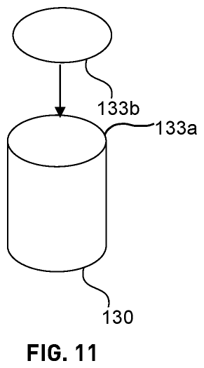

It is contemplated that the cassette 120 may include one or more mechanisms that facilitates exposure of the microtubule 130 to the user's saliva. FIG. 11 illustrates a configuration of a microtubule 130 in which the collection portion 132 includes an opening 133a, and a covering member 133b positioned over the opening 133a. According to an aspect, the covering member 133b may be removed from the opening when the cassette is coupled to the mouthpiece.

According to an aspect, the covering member 133b may be an opaque pressure sensitive adhesive (PSA) or strip that is removed, stripped away or torn away from a surface of the cassette when the cassette 120 is coupled to the oral appliance 110. Alternatively, the covering member 133b may include a see through pressure sensitive adhesive or strip. According to an aspect, the covering member 133b includes a material that is dissolved by the presence of moisture, such as the user's saliva, when the oral appliance 110 is positioned in the user's oral cavity.

FIG. 12 illustrates a system 100 in which the cassette 120 includes a microtubule 130 for collecting a sample of saliva to be tested, a light transmitter 135 for transmitting light through the sample, and a detector 140 to test for the presence of a target molecule 101 in the sample. In this configuration, the oral appliance 110 may, via a communication means 170, communicate the results of the test to a mobile device 200.

The oral appliance 110 may include its own power supply (not shown). The power supply may include a battery. While it is contemplated that the battery is rechargeable, it may be a disposable battery. The battery may be configured to provide power to at least one of light transmitter 135 and the detector 140. According to an aspect, the battery provides power to a sensor 122 provided in the oral appliance 110. The oral appliance 110 may further include power amplifiers configured to reduce power requirements of the oral appliance 110, thereby helping to conserve life of the rechargeable battery.

This disclosure, in various embodiments, configurations and aspects, includes components, methods, processes, systems, and/or apparatuses as depicted and described herein, including various embodiments, sub-combinations, and subsets thereof. This disclosure contemplates, in various embodiments, configurations and aspects, the actual or optional use or inclusion of, e.g., components or processes as may be well-known or understood in the art and consistent with this disclosure though not depicted and/or described herein.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

In this specification and the claims that follow, reference will be made to a number of terms that have the following meanings. The terms “a” (or “an”) and “the” refer to one or more of that entity, thereby including plural referents unless the context clearly dictates otherwise. As such, the terms “a” (or “an”), “one or more” and “at least one” can be used interchangeably herein. Furthermore, references to “one embodiment”, “some embodiments”, “an embodiment” and the like are not intended to be interpreted as excluding the existence of additional embodiments that also incorporate the recited features. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Terms such as “first,” “second,” “upper,” “lower” etc. are used to identify one element from another, and unless otherwise specified are not meant to refer to a particular order or number of elements.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur—this distinction is captured by the terms “may” and “may be.”

As used in the claims, the word “comprises” and its grammatical variants logically also subtend and include phrases of varying and differing extent such as for example, but not limited thereto, “consisting essentially of” and “consisting of.” Where necessary, ranges have been supplied, and those ranges are inclusive of all sub-ranges therebetween. It is to be expected that the appended claims should cover variations in the ranges except where this disclosure makes clear the use of a particular range in certain embodiments.

The terms “determine”, “calculate” and “compute,” and variations thereof, as used herein, are used interchangeably and include any type of methodology, process, mathematical operation or technique.

This disclosure is presented for purposes of illustration and description. This disclosure is not limited to the form or forms disclosed herein. In the Detailed Description of this disclosure, for example, various features of some exemplary embodiments are grouped together to representatively describe those and other contemplated embodiments, configurations, and aspects, to the extent that including in this disclosure a description of every potential embodiment, variant, and combination of features is not feasible. Thus, the features of the disclosed embodiments, configurations, and aspects may be combined in alternate embodiments, configurations, and aspects not expressly discussed above. For example, the features recited in the following claims lie in less than all features of a single disclosed embodiment, configuration, or aspect. Thus, the following claims are hereby incorporated into this Detailed Description, with each claim standing on its own as a separate embodiment of this disclosure.

Advances in science and technology may provide variations that are not necessarily express in the terminology of this disclosure although the claims would not necessarily exclude these variations.

1. A system for detecting target molecules in a patient's saliva, the system comprising:
an oral appliance configured to be positioned in an oral cavity of the patient and removably secured to dentition of the patient;
a cassette removably coupled to the oral appliance, the cassette comprising a microtubule including a collection portion; and
a detector coupled to the oral appliance, wherein the detector is configured to:
transmit light through the cassette;
measure an amount of light transmitted through the cassette;
determine an absorption or scattering amount of the light transmitted through the cassette; and
determine whether the target molecules are present in the patient's saliva based on the absorption or scattering amount of the light.

2. The system of claim 1, wherein the microtubule further comprises:
a treatment portion spaced apart from the collection portion, the treatment portion comprising a plurality of nanoparticles.

3. The system of claim 2, wherein the plurality of nanoparticles comprise at least one of gold, silver or silica.

4. The system of claim 2, wherein the plurality of nanoparticles are configured to bond to the target molecules to form a plurality of target nanoparticles.

5. The system of claim 4, wherein the microtubule further comprises:
a testing portion, wherein
the testing portion is spaced apart from the collection portion and the treatment portion, and
the target nanoparticles are housed in the testing portion.

6. The system of claim 5, wherein the testing portion is aligned with the detector so that the light is transmitted through the target nanoparticles.

7. The system of claim 1, wherein the patient's saliva is transported along at least a portion of a length of the cassette via capillary movement or hydrostatic pressure.

8. The system of claim 1, wherein the detector comprises a spectrophotometer.

9. The system of claim 1, wherein the target molecule is an amino acid.

10. The system of claim 1, wherein the collection portion comprises:
an opening; and
a covering member positioned over the opening,
wherein the covering member is removed from the opening when the cassette is coupled to the oral appliance.

11. A system for detecting target molecules in a patient's saliva, the system comprising:
a mouthpiece configured to be positioned in an oral cavity of the patient and removably secured to dentition of the patient;
a cassette removably coupled to the mouthpiece, the cassette comprising a microtubule including a collection portion; and
a detector in wireless communication with the cassette, wherein:
the cassette, when removed from the mouthpiece, is configured to be positioned on a testing surface of the detector, and
the detector is configured to:
transmit light through the cassette;
measure an amount of light transmitted through the cassette;
determine an absorption or scattering amount of the light transmitted through the cassette; and
determine whether the target molecules are present in the patient's saliva based on the absorption or scattering amount of the light.

12. The system of claim 11, wherein the microtubule further comprises:
a testing portion for housing collected saliva.

13. The system of claim 12, wherein the testing portion is alignable with the testing surface of the detector so that the light is transmitted through the collected saliva.

14. The system of claim 12, wherein:
the testing portion is spaced apart from the collection portion, and
the patient's saliva is transported from the collection portion to the testing portion via capillary movement or hydrostatic pressure.

15. The system of claim 12, wherein the testing portion extends along at least a portion of a length of the microtubule.

16. The system of claim 11, wherein the collection portion comprises:
an opening; and
a covering member positioned over the opening, the covering member comprising at least one of a dissolvable material or a removable material.

17. A system for detecting target molecules in a patient's saliva, the system comprising:
an oral appliance;
a cassette removably coupled to the oral appliance, the cassette comprising:
a microtubule comprising a collection portion;
a light transmitter configured to transmit light through the microtubule; and
a detector, wherein the detector is configured to:
measure an amount of light transmitted through the microtubule;
determine an absorption or scattering amount of the light transmitted through the microtubule; and
determine whether the target molecules are present in the patient's saliva based on the absorption or scattering amount of the light.

18. The system of claim 17, wherein:
the microtubule further comprises a testing portion for housing collected saliva, and
the light transmitter is configured to transmit the light through the testing portion.

19. The system of claim 18, wherein:
the testing portion is spaced apart from the collection portion, and
the patient's saliva is transported from the collection portion to the testing portion via capillary movement or hydrostatic pressure.

20. The system of claim 17, wherein the collection portion comprises:
an opening; and
a covering member positioned over the opening,
wherein the covering member is automatically removed from the opening once the oral appliance is positioned in an oral cavity of the patient.
